(19) United States
(12) Patent Application Publication
(10) Pub. No.: US 2007/0129722 A1
(43) Pub. Date: Jun. 7, 2007

(54) SWIVEL DEVICE FOR IMPROVED SURGICAL SMOKE EVACUATION

(76) Inventor: Ioan Cosmescu, Phoenix, AZ (US)

Correspondence Address: SNELL & WILMER L.L.P. (Main), 400 EAST VAN BUREN, ONE ARIZONA CENTER, PHOENIX, AZ, 85004-2202, US

(21) Appl. No.: 11/164,712

(22) Filed: Dec. 2, 2005

(51) Int. Cl. A61M 37/00; A61B 18/18

(52) U.S. Cl. 606/42; 604/26

(57) ABSTRACT

A swivel device includes an improved connection between an electro-surgical unit (ESU) pencil's smoke evacuation system and a vacuum tube. The swivel device includes a fixed member attached to a rotating member. The rotating member allows the stiff vacuum tube to twist and coil freely while preventing forced movement of the fixed member or the ESU pencil. The swivel device can be added to existing ESU pencils with smoke evacuation systems or it can be built into ESU pencils with smoke evacuation systems.

### CROSS-REFERENCE TO RELATED APPLICATIONS

This application is related to patent application Ser. No. 08/500,045, filed Jul. 10, 1995, now U.S. Pat. No. 5,693,044, which is a continuation-in-part of application Ser. No. 07/989,238, filed Dec. 11, 1992, now abandoned.

### FIELD OF INVENTION

The present invention relates generally to a swivel apparatus. More specifically, the present invention relates to a swivel apparatus for attachment to or incorporation with the smoke evacuation system of an electro-surgical unit (ESU) pencil, and methods therefore.

### DESCRIPTION OF THE RELATED ART

The use of ESU pencils for cutting tissue and coagulating blood vessels in surgical procedures is well known. When an ESU pencil is used for cutting or coagulation, smoke is produced. In the past, when a surgeon wanted to evacuate this smoke from the surgical field, the surgeon or an assistant had to hold a plastic suction wand, connected to vacuum tubing, near the site of smoke production. This became cumbersome in many surgical procedures, because two hands were required—one operating the ESU pencil and the other holding the suction device—and because the suction wand often obscured the surgeon's view of the surgical field. Therefore, smoke evacuation devices were developed which were either incorporated directly into the design of an ESU pencil or were attachable to an ESU pencil.

Built-in or attachable smoke evacuation devices have also proved problematic, however. Like the suction wand, these devices must be connected to a vacuum source via a vacuum tube. The vacuum tubing generally used is stiff, corrugated, rubber tubing. Due to its stiffness, the tubing often coils in such a way that it twists the ESU pencil in the hand of the surgeon. This twisting of the ESU pencil is problematic in delicate surgical procedures and often requires the surgeon to stop frequently to uncoil the tubing. Therefore, more flexible vacuum tubing has been tried. However, this tubing is either too soft, so that it collapses under suction, or too narrow, so that it obstructs airflow. These various problems with vacuum tubing have led many surgeons to not use built-in or attached smoke evacuation devices on ESU pencils. But the only alternative is the separate suction wand, which is often too cumbersome or visually obstructing.

Therefore, there existed a need for an improved connection between an ESU pencil's built-in or attached smoke evacuation system and a vacuum tube. Such a connection would need to allow the stiff, corrugated, vacuum tube to twist without turning the ESU pencil in the surgeon's hand. The connection would also have to remain airtight, so that the force of the smoke evacuation system's vacuum was not reduced.

### SUMMARY OF INVENTION

The present invention is directed to a novel swivel apparatus that satisfies these needs. A preferred version of the swivel apparatus comprises a fixed member coupled to a rotating member. The distal end of the fixed member is rigidly attached to the smoke evacuation system of an ESU pencil and the proximal end of the fixed member fits inside the distal end of the rotating member. The distal end of the rotating member fits around the proximal end of the fixed member such that the rotating member rotates freely around the fixed member. The proximal end of the rotating member then fits inside the distal end of a vacuum tube. Thus, the rotating member allows the vacuum tube to rotate without twisting the fixed member or the ESU pencil.

Furthermore, the extreme proximal end of the fixed member is slightly inverted. This circumferential inversion fits directly against a circumferential inversion near the lengthwise center of the rotating member. When a vacuum is applied, via the vacuum tube, this inverted surface creates an increased vacuum force which presses the inverted proximal end of the fixed member against the inverted center of the rotating member, creating a virtually airtight seal. This seal prevents leakage of air from the swivel apparatus and, thus, maintains the power and efficiency of the vacuum.

Therefore, when no vacuum source is applied, the rotating member rotates freely around the fixed member, allowing the vacuum tube to rotate without rotating the ESU pencil. When the vacuum is applied, the rotating member rotates less freely around the fixed member, but the swivel apparatus becomes virtually airtight so that no vacuum force or efficiency is lost.

To increase the applicability of the swivel apparatus, the fixed swivel means may be a separate piece that is attachable to an ESU pencil integrated smoke evacuation system or smoke evacuation shroud attachment and it may also be a permanently incorporated part of an ESU pencil integrated smoke evacuation system or smoke evacuation shroud attachment.

### BRIEF DESCRIPTION OF THE DRAWINGS

A more complete understanding of the present invention may be derived by referring to the detailed description and claims when considered in connection with the following illustrative Figures, which may not be to scale. In the following Figures, like reference numbers refer to similar elements throughout the Figures.

FIG. 1 is a perspective view of one embodiment of a swivel device according to the present invention, for attachment to an ESU pencil with integrated smoke evacuation system or to an ESU pencil smoke evacuation shroud attachment.

FIG. 2 is a cross-sectional view of the fixed member of the swivel device in FIG. 1.

FIG. 3 is a cross-sectional view of the rotating member of the swivel device in FIG. 1.

FIG. 4A is a perspective view of an ESU pencil with an integrated smoke evacuation system and another embodiment of the swivel device which is permanently incorporated into the proximal end of the ESU pencil.

FIG. 4B is a perspective view of the proximal end of the ESU pencil of FIG. 4A, but without an incorporated swivel device.

FIG. 4C is a side view of the ESU pencil with integrated smoke evacuation system and permanently incorporated swivel device in FIG. 4A, with a partial cross-sectional view of the fixed member and a cross-sectional view of a detached rotating member.

FIG. 5A is a perspective view of an ESU pencil with a smoke evacuation shroud attachment and another embodiment of the swivel device which is permanently incorporated into the proximal end of the smoke evacuation shroud attachment.

FIG. 5B is a perspective view of the proximal end of the ESU pencil of FIG. 5A, but without an incorporated swivel device.

### DETAILED DESCRIPTION

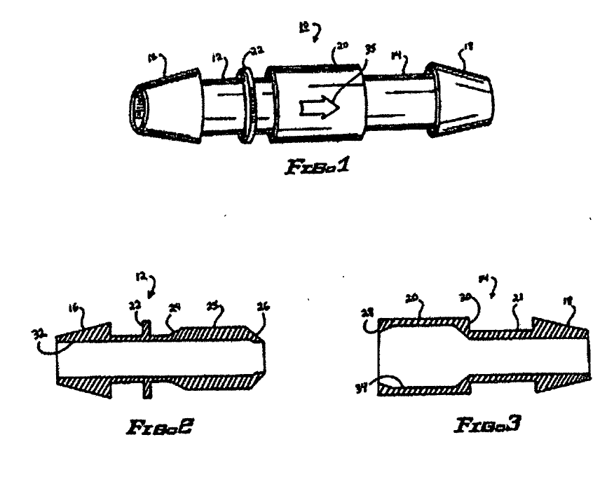

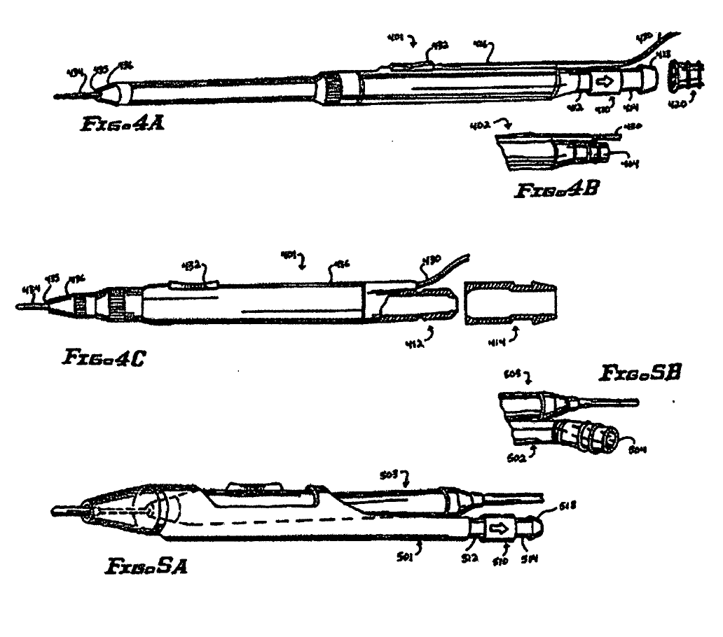

The present invention is directed to a swivel device for attachment to or incorporation with an electro-surgical (ESU) pencil smoke evacuation system. With reference to the drawings, FIGS. 1, 2 and 3 depict one embodiment of a swivel device 10 for attachment to an ESU pencil with an integrated smoke evacuation system 402, as shown in FIG. 4B, or to a smoke evacuation shroud attachment for an ESU pencil 502, as shown in FIG. 5B. FIG. 4B depicts the proximal end of one embodiment of an ESU pencil 402 with an integrated smoke evacuation system. FIG. 5B depicts the proximal end of another embodiment of an ESU pencil 503 with a smoke evacuation shroud attachment 502.

The swivel device 10 comprises a fixed member 12 and a rotating member 14. FIG. 1 shows a perspective view of an assembled swivel device 10, with fixed member 12 and rotating member 14. FIGS. 2 and 3 show cross-sectional views of the fixed member 12 and the rotating member 12 of the swivel device 10, respectively.

The exhaust port connector 16 of the fixed member 12 connects directly to the exhaust port 404 of an ESU pencil with an integrated smoke evacuation system 402 or to the exhaust port 504 of a smoke evacuation shroud attachment 502. The exhaust port connector 16 is defined as the distal end of the swivel device 10. The vacuum tube connector 18 of the rotating member 14, which connects directly to a vacuum tube 420, is defined as the proximal end of the swivel device 10. Hereafter in the specification, this definition is the reference for the use of the terms “distal end” and “proximal end” with respect to each element of the swivel device 10.

The exhaust port connector 16 at the distal end of the fixed member 12 is tapered to fit into the exhaust port of an ESU pencil 404 or smoke evacuation shroud attachment 504, creating an airtight connection. Threaded engagement or other means of connecting well known in the art are also acceptable to connect the exhaust port connector 16 to the exhaust port 404/504. The fixed member also has an exhaust port abutment 22, against which the exhaust port 404/504 may abut to prevent over-insertion of the fixed member 12 into the exhaust port 404/504. Just proximal to the exhaust port abutment 22, the external surface of the fixed member 12 has a ridge 24 where the outer diameter of the fixed member 12 becomes larger. This larger-outer-diameter-portion 25 of the fixed member 12 has an outer diameter and length which allows it to fit within the widened distal portion 20 of the rotating member 14. The proximal end 26 of the fixed member 12 is inverted to fit against a similar inversion 30 near the lengthwise center of the rotating member 14. As shown in FIG. 2, the internal surface of the fixed swivel means 32 is preferably a straight cylinder, except at the inverted proximal end 26.

The distal end 28 of the rotating member 14 is inverted, to fit around the ridge 24 of the fixed member 12. The widened distal portion 20 of the rotating member 14 has an internal diameter and length which allows it to fit and rotate freely around the larger-outer-diameter-portion 25 of the fixed member 12. The inverted distal end 28 on the interior surface of the rotating member 14 and central inversion 30 on the exterior surface of the rotating member 14, along with the ridge 24 and inverted proximal end 26 on the external surface of the fixed member 12, allow the fixed member 12 and the rotating member 14 to be coupled together without sliding in a lengthwise direction in relation to one another. The proximal end of the rotating member 14 also has a vacuum tube connector 18, which is tapered to fit into the distal end of a vacuum tube 420, creating an airtight connection. Threaded engagement or other means of connecting well known in the art are also acceptable to connect the vacuum tube connector 18 to the vacuum tube 420. As shown in FIG. 3, the internal surface 34 of the rotating member 14 is a straight cylinder with a widened internal diameter at the distal portion 20, a narrowed diameter at the proximal portion 21, and internal inversions of the widened distal portion 20 at the distal end 28 and near the lengthwise center 30 of the rotating member 14. The external surface of the widened portion 20 of the rotating member 14 has an arrow 35 (see FIG. 1), which may be printed or engraved in any practical method and which points from the distal end to the proximal end of the swivel device 10, to prevent improper direction of attachment to the exhaust port 404/504 and the vacuum tube 420.

FIG. 4A depicts an ESU pencil with an integrated smoke evacuation system 401 and another embodiment of the swivel device 410 which is permanently incorporated into the proximal end of the ESU pencil 402 shown in FIG. 4B. FIG. 4C is a side view of the ESU pencil shown in FIG. 4A, with a partial cross-sectional view of the fixed member 412 and a cross-sectional view of a detached movable member 414. The ESU pencil with integrated smoke evacuation system 401 (but without any swivel device) is described in full detail in patent application Ser. No. 500,045, filed Jul. 10, 1995, now U.S. Pat. No. 5,693,044, which is a continuation-in-part of application Ser. No. 989,238, filed Dec. 11, 1992, now abandoned.

The embodiment of the swivel device 410 in FIGS. 4A and 4C is nearly identical to that of the swivel 10 in FIGS. 1, 2 and 3, with the following exceptions. The fixed member 412 is permanently built into the proximal end of the handpiece 416 of the ESU pencil 401. Thus, the fixed member 412 does not contain an exhaust port connector 16 or an exhaust port abutment 22. Otherwise, the fixed member 412 is identical to the fixed member 12 in FIGS. 1, 2 and 3. The rotating member 414 is identical to the rotating member 14 of the swivel device 10 in FIGS. 1, 2 and 3.

FIG. 5A depicts an ESU pencil 503 with a smoke evacuation shroud attachment 501 and another embodiment of the swivel device 510 which is permanently incorporated into the proximal end of the smoke evacuation shroud attachment 501. The ESU pencil 503 with smoke evacuation shroud attachment 501 (but without any swivel device) is described in full detail in patent application Ser. No. 500,045, filed Jul. 10, 1995, now U.S. Pat. No. 5,693,044, which is a continuation-in-part of application Ser. No. 989,238, filed Dec. 11, 1992, now abandoned. The embodiment of the swivel device 510 in FIG. 5A is identical to that of the swivel device 410 in FIG. 4A except that the fixed member 512 is permanently built into the smoke evacuation shroud attachment 501, rather than an ESU pencil handpiece 416.

Referring to FIG. 4A, before beginning a surgical procedure, the vacuum tube connector 418 of the rotating member 414 is connected to the vacuum tube 420 which is connected to a vacuum source (not shown) and the power cord 430 for the ESU pencil 401 is connected to a power source (not shown). When desired, the surgeon then uses the hand switch 432 to send radio frequency energy to the ESU pencil's 401 electrode 434, for cutting and coagulation. When the vacuum source is activated, smoke and debris from cutting and coagulation are sucked into the aperture 435 at the distal end of the nozzle 436 of the ESU pencil's 401 smoke evacuation system. The smoke and debris then travel through the smoke evacuation system, the swivel device 410, and the vacuum tube 420, to a vacuum canister (not shown). The operation of the ESU pencil with integrated smoke evacuation system 401 is described in full detail in patent application Ser. No. 500,045, filed Jul. 10, 1995, now U.S. Pat. No. 5,693,044, which is a continuation-in-part of application Ser. No. 989,238, filed Dec. 11, 1992, now abandoned.

When the vacuum source is not activated, the rotating member 414 is free to rotate around the fixed member 412, thus allowing the vacuum tube 420 to twist and coil without moving the ESU pencil handpiece 416 in the surgeon's hand. Referring to FIGS. 2 and 3, when the vacuum source is activated, the inverted proximal end 26 of the fixed member 12 and the inverted surface 30 of the rotating member 14 create an increased vacuum force at the site of the inversions. This increased vacuum force presses the inverted proximal end 26 of the fixed member 12 more tightly against the inverted surface 30 of the rotating member 14, creating a more airtight seal but also decreasing the free rotation of the rotating member 14. Some vacuum sources are automatic, such that they are activated whenever the surgeon presses the hand switch 432 and deactivated whenever the hand switch 432 is not being pressed. Other vacuum sources must be activated and deactivated manually. The swivel device 10 of the present invention will operate with any type of vacuum source. However, the swivel device 10 may be easiest to use with automatically activated and deactivated vacuum sources, since the rotating member 414 will rotate freely whenever the surgeon releases his finger from the hand switch 432.

The increased vacuum force created by the inverted proximal end 26 of the fixed member 12 and the surface inversion 30 of the rotating member 14, as described above, is essential for proper function of the swivel device 10. This increased vacuum force creates a virtually airtight seal, which prevents leakage of air from the swivel device 10 and, thus, maintains the ability of the smoke evacuation system to clear smoke and debris from the site of cutting and coagulation.

Referring to FIG. 5A, the operation of the incorporated swivel device 510 is essentially the same as the operation of the incorporated swivel device 410 described in FIG. 4A. The only difference is that the fixed member 512 is permanently incorporated into the proximal end of the smoke evacuation shroud attachment 501, rather than the proximal end of an ESU pencil with integrated smoke evacuation system 401. The operation of the ESU pencil 503 with smoke evacuation shroud attachment 501 is described in full detail in patent application Ser. No. 500,045, filed Jul. 10, 1995, now U.S. Pat. No. 5,693,044, which is a continuation-in-part of application Ser. No. 989,238, filed Dec. 11, 1992, now abandoned.

Referring to FIGS. 1, 2, 3, 4B and 5B, the operation of this embodiment of the swivel device 10 is essentially the same as that described in FIGS. 4A and 5A. The only difference is that the swivel device 10 is a separate piece which must be attached to the exhaust port 404 of an ESU pencil with integrated smoke evacuation system 402 or to the exhaust port 504 of a smoke evacuation shroud attachment 502.

The present invention has been described above with reference to preferred embodiments. However, those skilled in the art, having read this disclosure, will recognize that changes and modifications may be made to the preferred embodiments without departing from the scope of the present invention. For example, the swivel device of the present invention may also be used or incorporated with laser or fiber optic surgical pencils that are employed along with suction devices to eliminate smoke and debris from the surgical site. These and other changes or modifications are intended to be included within the scope of the present invention, as expressed in the following claims.

What is claimed is:

1. A swivel device comprising, in combination:
a fixed member capable of removable attachment to an exhaust port of at least one of an ESU pencil with an integrated smoke evacuation system or an ESU pencil smoke evacuation shroud attachment; and
a rotating member coupled to the fixed member for connecting the fixed member to a vacuum tube and for allowing rotation of the vacuum tube without rotation of at least one of the ESU pencil with an integrated smoke evacuation system or the ESU pencil smoke evacuation shroud attachment.

2. The apparatus of claim 1 wherein the fixed member comprises a hollow tubular member having an open distal end and an open proximal end.

3. The apparatus of claim 2 wherein the distal end has a tapered external surface for fitting within the exhaust port.

4. The apparatus of claim 2 wherein the hollow tubular member has an exhaust port abutment near the distal end for preventing over-insertion of the fixed member into the exhaust port.

5. The apparatus of claim 2 wherein the hollow tubular member has a proximal portion that fits precisely within a distal portion of the rotating member, for preventing lengthwise movement of the fixed member within the rotating member.

6. The apparatus of claim 2 wherein the hollow tubular member has a smooth cylindrical internal surface for conducting smoke and debris out of at least one of the ESU pencil with an integrated smoke evacuation system or the ESU pencil smoke evacuation shroud attachment.

7. The apparatus of claim 2 wherein the proximal end has an inverted surface for creating a virtually airtight connection with an inverted surface near a lengthwise center of the rotating member when a vacuum source is applied.

8. The apparatus of claim 1 wherein the rotating member comprises a hollow tubular member having an open distal end and an open proximal end.

9. The apparatus of claim 8 wherein the hollow tubular member has a distal portion with an outer diameter that fits precisely around a proximal portion of the fixed member for allowing rotation of the rotating member around the fixed member while preventing lengthwise movement of the rotating member on the fixed member.

10. The apparatus of claim 8 wherein the hollow tubular member has an inverted surface near a lengthwise center for creating a virtually airtight connection with an inverted proximal end of the fixed member when a vacuum source is applied.

11. The apparatus of claim 8 wherein the hollow tubular member has a smooth cylindrical internal surface for conducting smoke and debris to the vacuum tube.

12. The apparatus of claim 8 wherein the proximal end has a tapered external surface for fitting within the vacuum tube.

13. A swivel device comprising, in combination:
a fixed member for permanent incorporation into a proximal end of at least one of an ESU pencil with an integrated smoke evacuation system or an ESU pencil smoke evacuation shroud attachment; and
a rotating member coupled to the fixed member for connecting the fixed member to a vacuum tube and for allowing rotation of the vacuum tube without rotation of at least one of the ESU pencil with an integrated smoke evacuation system or the ESU pencil smoke evacuation shroud attachment.

14. The apparatus of claim 13 wherein the fixed member comprises a first hollow tubular member having a first open distal end and a first open proximal end and the rotating member comprises a second hollow tubular member having a second open distal end and a second open proximal end.

15. The apparatus of claim 14 wherein the first hollow tubular member has an outer proximal portion that fits precisely within an inner distal portion of the rotating member, for preventing lengthwise movement of the fixed member within the rotating member.

16. The apparatus of claim 14 wherein the first open proximal end has an inverted surface for creating a virtually airtight connection with an inverted surface near a lengthwise center of the rotating member when a vacuum source is applied.

17. The apparatus of claim 15 wherein the second open distal end of the second hollow tubular member has a diameter that fits precisely around the outer proximal portion of the fixed member for allowing rotation of the rotating member around the fixed member while preventing lengthwise movement of the rotating member on the fixed member.

18. The apparatus of claim 16 wherein the second hollow tubular member has an inverted surface near a lengthwise center for creating a virtually airtight connection with the inverted first open proximal end of the fixed member when a vacuum source is applied.

19. The apparatus of claim 14 wherein the second open proximal end has a tapered external surface for fitting within the vacuum tube.

20. A method for making a swivel-based surgical pencil comprising the steps of:
a) attaching a distal end of a fixed member to an exhaust port of at least one of an ESU pencil with an integrated smoke evacuation system or an ESU pencil smoke evacuation shroud attachment;
b) attaching a proximal end of the fixed member to a distal end of a rotating member; and
c) attaching a proximal end of the rotating member to a distal end of a vacuum tube.
